(19) United States
(12) Patent Application Publication
(10) Pub. No.: US 2005/0065574 A1
(43) Pub. Date: Mar. 24, 2005

(54) Methods of affecting hypothalamic-related conditions

(76) Inventor: Ali Rezai, Bratenhal, OH (US)

Correspondence Address: KENYON & KENYON, 1500 K STREET, N.W., SUITE 700, WASHINGTON, DC, 20005, US

(21) Appl. No.: 10/900,301

(22) Filed: Jul. 28, 2004

Related U.S. Application Data
(63) Continuation-in-part of application No. PCT/US03/02847, filed on Jan. 31, 2003.
(60) Provisional application No. 60/353,697, filed on Feb. 1, 2002.
(60) Provisional application No. 60/567,441, filed on May 4, 2004.

(51) Int. Cl. A61N 1/18

(52) U.S. Cl. 607/45

(57) ABSTRACT

The present invention relates to a method of affecting a hypothalamic-related condition by electrically and/or chemically stimulating the hypothalamus. Also provided are methods of stimulating a hypothalamic-related target site by responding to a sensor signal relating to a physiological activity of the body associated with the hypothalamic-related condition desired to be affected. The present invention also describes a method of directly or indirectly modulating hormones synthesized or released by the hypothalamus to affect hypothalamic-related conditions involving hormonal function, dysfunction or imbalance.

### CROSS-REFERENCE TO RELATED APPLICATIONS

This application is a continuation-in-part of PCT Application No. PCT/US03/02847, filed on Jan. 31, 2003, which claims the benefit of Provisional U.S. Application No. 60/353,697, filed Feb. 1, 2002, all of which are incorporated by reference herein. This application also claims the benefit of U.S. Provisional No. 60/567,441, filed on May 4, 2004, which is incorporated by reference herein.

### BACKGROUND OF THE INVENTION

The hypothalamus is a central neurological structure composed of several sub-components that control a wide array of physiological functions in the human body. In particular, the hypothalamus modulates numerous fundamental body functions such as heart rate, body temperature, blood pressure, fluid and electrolyte balance, sleep, and food intake. In addition, the hypothalamus with the pituitary gland, is involved in endocrine activity such as the development of secondary sex characteristics and ovulation. The hypothalamus communicates with the pituitary gland by either secreting hormones that are released into the blood and travel to the anterior lobe of the pituitary where such hormones exert their effects, or the hormones travel in neurons to the posterior lobe of the pituitary where they are released into circulation. The hypothalamus also plays a role in regulating complex moods, such as anger, fear, sexual drive, placidity, and fatigue. Because of the hypothalamus' diverse and multiple roles, aberrant functioning of the hypothalamus or of structures and pathways that communicate with the hypothalamus can contribute to such varied conditions and disorders as arrhythmia, acromegaly and infertility.

For many of these conditions, current therapies generate intolerable side effects, require repeated administration of treatment, or are simply ineffective in a subset of patients. For example, in the treatment of acromegaly, a disorder caused by prolonged overproduction of growth hormone by the pituitary gland and characterized by the abnormal growth of bone and cartilage, currently available drugs are effective in lowering growth hormone secretion in less than half the patient population or must be injected subcutaneously every 8 hours for effective treatment.

Furthermore, many of these hypothalamic-related conditions are prevalent and therefore effective treatment is particularly desirable. For example, nearly 6.1 million Americans suffer from infertility, which accounts for approximately ten percent of the reproductive age population, and reports indicate that one in six of all couples seek medical help because of infertility. Despite the desire for treatment, current fertility techniques are inefficacious for many patients. For example, reports indicate that in vitro fertilization, at best, is successful in only 25% of recipients. Therefore, there is an unmet need in the art for an effective method of treating hypothalamic-related conditions that provides a viable alternative for patients unresponsive to or dissatisfied with current therapy options.

### SUMMARY OF THE INVENTION

The present invention relates to methods of affecting a hypothalamic-related condition by electrically and/or chemically stimulating a hypothalamic-related target site to modulate the target site. Specifically, the present invention relates to implanting a stimulator, which can be either an electrode or catheter, into a target site of a hypothalamic-associated circuitry, a hypothalamus, a division of a hypothalamus, or a nucleus of a hypothalamus to electrically and/or chemically stimulate the target site to modulate the target site to affect the hypothalamic-related condition.

In particular, one embodiment of the present invention provides a method of affecting a hypothalamic-related condition, other than an eating disorder, by implanting a stimulator in a target site of a hypothalamic-associated circuitry and providing a stimulation signal to the stimulator to stimulate the target site to affect the hypothalamic-related condition.

In another embodiment, the present invention provides a method of affecting a hypothalamic-related condition, other than an eating disorder, by implanting a stimulator in a target site of a hypothalamus and providing a stimulation signal to the stimulator to stimulate the target site to affect the hypothalamic-related condition.

In yet another embodiment, the present invention provides a method of affecting a hypothalamic-related condition, other than an eating disorder, by implanting a stimulator in a target site of a division of a hypothalamus and providing a stimulation signal to the stimulator to stimulate the target site to affect the hypothalamic-related condition.

In a further embodiment, the present invention provides a method of affecting a hypothalamic-related condition, other than an eating disorder, by implanting a stimulator in a nucleus of a hypothalamus and providing a stimulation signal to the stimulator to stimulate the nucleus to affect the hypothalamic-related condition.

In another embodiment, the present invention provides a method of affecting an eating disorder by implanting a stimulator in a target site and providing a stimulation signal to the stimulator to stimulate the target site to affect the eating disorder, wherein the target site is selected from the group consisting of the tuberoinfundibular tract, dorsal longitudinal fasciculus, nucleus ambiguus, and fornix, periaqueductal gray.

The present invention also provides a method of affecting a hypothalamic-related condition by implanting a stimulator in communication with a hypothalamic-related target site, detecting a physiological activity of the body associated with the hypothalamic-related condition to generate a sensor signal, and providing a stimulation signal to the stimulator in response to the sensor signal to affect the hypothalamic-related condition. The hypothalamic-related target site can be a target site of the hypothalamic-associated circuitry, the hypothalamus, a division of the hypothalamus, or a nucleus of the hypothalamus.

The present invention also provides a method of affecting a hypothalamic-related condition by implanting a stimulator in a target site of a hypothalamus or pituitary gland, and providing a stimulation signal to the stimulator to stimulate the target site to modulate the synthesis or release of a hypothalamic or pituitary product, such as a hormone or neuropeptide.

### BRIEF DESCRIPTION OF THE FIGURES AND TABLES

FIG. 1 is a cross-sectional view of the brain showing placement of a stimulator to practice a method according to the present invention.

Table I provides components of a hypothalamic-associated circuitry.

Table II provides target sites of the hypothalamic-associated circuitry to affect corresponding hypothalamic-related conditions.

Table III provides components of a hypothalamus.

Table IV provides target sites of the hypothalamus to affect corresponding hypothalamic-related conditions.

Table V provides divisions of the hypothalamus.

Table VI provides target divisions of the hypothalamus to affect corresponding hypothalamic-related conditions.

Table VII provides nuclei of the hypothalamus.

Table VIII provides nuclei of the hypothalamus to affect corresponding hypothalamic-related conditions.

Table IX provides hypothalamic and pituitary products.

Table X provides hypothalamic and pituitary products and hypothalamic-related conditions affected by modulation of such products.

Table XI provides target sites of the hypothalamus to affect the modulation of corresponding hypothalamic and pituitary products.

### DETAILED DESCRIPTION OF THE INVENTION

The present invention relates to methods of affecting a hypothalamic-related condition to regulate, prevent, treat, alleviate the symptoms of and/or reduce the effects of such hypothalamic-related condition. By “hypothalamic-related condition” is generally meant a condition, disease, disorder, function, or abnormality that is directly or indirectly modulated by the hypothalamus. Hypothalamic-related conditions according to the present invention include pain, hypertension, congestive heart failure, hyperthyroidism, hypothyroidism, acromegaly, prolactinomas, psychogenic polydipsia, uncontrollable phobias, body temperature regulation, ovulation, infertility, aggression and disorders of the eye, lacrimary and salivary glands, liver, heart, esophagus, lungs, stomach (gastrointestinal), pancreas, small intestine, large intestine, rectum, bladder, or reproductive organs. In the case of affecting pain, such pain may be the result of a condition resulting from one or more medical conditions such as, for example, migraine headaches, including migraine headaches with aura, migraine headaches without aura, menstrual migraines, migraine variants, atypical migraines, complicated migraines, hemiplegic migraines, transformed migraines, and chronic daily migraines; episodic tension headaches; chronic tension headaches; analgesic rebound headaches; episodic cluster headaches; chronic cluster headaches; cluster variants; chronic paroxysmal hemicrania; hemicrania continua; post-traumatic headache; post-traumatic neck pain; post-herpetic neuralgia involving the head or face; pain from spine fracture secondary to osteoporosis; arthritis pain in the spine; headache related to cerebrovascular disease and stroke; headache due to vascular disorder; reflex sympathetic dystrophy; cervicalgia; glossodynia; carotidynia; cricoidyna; otalgia due to middle ear lesion; gastric pain; sciatica; maxillary neuralgia; laryngeal pain; myalgia of neck muscles; trigeminal neuralgia; post-lumbar puncture headache; low cerebro-spinal fluid pressure headache; temporomandibular joint disorder; atypical facial pain; ciliary neuralgia; paratrigeminal neuralgia; petrosal neuralgia; Eagle's syndrome; idiopathic intracranial hypertension; orofacial pain; myofascial pain syndrome involving the head, neck, and shoulder; chronic migraneous neuralgia; cervical headache; paratrigeminal paralysis; sphenopalatine ganglion neuralgia; carotidynia; Vidian neuralgia; and causalgia. Other examples of hypothalamic-related conditions will be readily appreciated by one of skill in the art and the present invention contemplates a method of affecting these hypothalamic-related conditions as well.

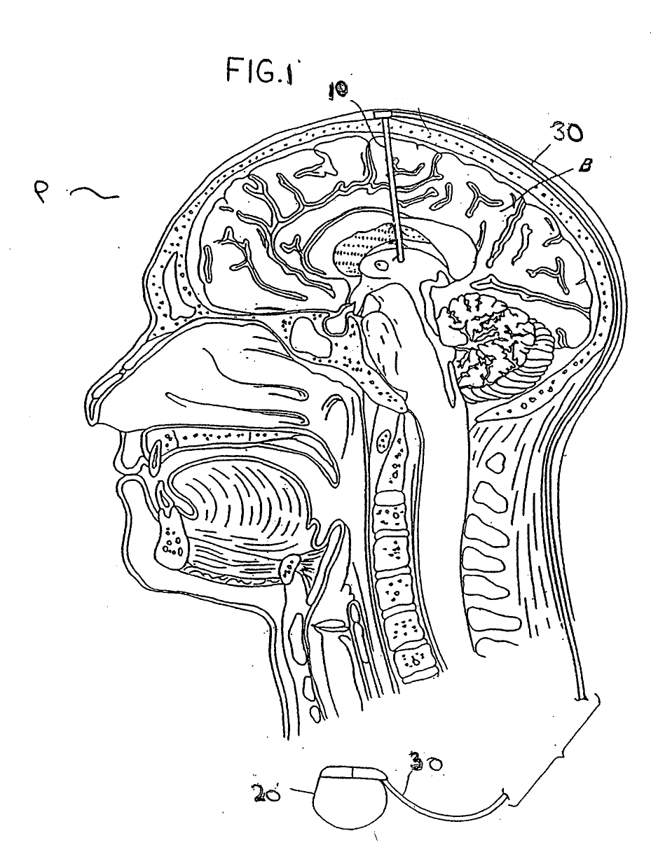

In general, the present invention provides for a method of affecting a hypothalamic-related condition by implanting a stimulator in a hypothalamic-related target site to modulate the target site such that the hypothalamic-related condition is affected. By “hypothalamic-related target site” is meant a target site of a hypothalamic-associated circuitry, a hypothalamus, a division of a hypothalamus, or a nucleus of hypothalamus, as described in greater detail herein. Referring to FIG. 1, in one example of a preferred mode of carrying out a method of the present invention, a stimulator 10, which can be either a catheter or electrode assembly, is implanted within a hypothalamic-related target site of brain B of a patient P. Stimulator 10 is, in turn, coupled to a stimulator controller 20, which is a pulse generator or drug pump, that generates electrical or chemical stimulation signals that are sent to stimulator 10 to electrically or chemically stimulate the hypothalamic-related target site. A connector 30, which is an insulated conductor in the case of electrical stimulation and an extension of a catheter in the case of chemical stimulation, couples stimulation controller 20 to stimulator 10. Stimulation controller 20 is, in turn, implanted in the abdomen, chest, or any other part of a patient P's body and is preferably in patient P's control or is a radio frequency controlled device operated by an external transmitter. In the case of a chemical delivery system where stimulator 10 is a catheter, stimulation controller 20 is preferably accessed subcutaneously such that a hypodermic needle can be inserted through the skin to inject a quantity of a chemical agent, such as a neuromodulation agent. The chemical agent is delivered from the stimulation controller 20 through a catheter port into the stimulator 10. Stimulation controller 20 may be a permanently implanted in patient P or only temporarily implanted such as the temporary neurostimulator described in co-pending U.S. Provisional No. 60/358,176.

With respect to the actual aspects of the methods of the present invention, in one embodiment, a method of treating a hypothalamic-related disorder including implanting a stimulator in a target site of a hypothalamic-associated circuitry and providing a stimulation signal to the stimulator to stimulate the target site to affect the hypothalamic-related condition. A hypothalamic-associated circuitry according to the present invention is the hypothalamus and all efferent and afferent structures and pathways that project to or from the hypothalamus. Referring to Table I, such afferent and efferent structures and pathways include the hypothalamus, limbic structures; nucleus solitarius; reticular formation; optic nerve; retina; optic chiasm; circumventricular organs; cranial nerve nuclei; midbrain raphe nuclei; various regions of the cerebral cortex; ventrolateral medulla; nucleus ambiguus; pituitary gland; and various tracts that run to or from the hypothalamus. Although not wishing to be bound by theory, preferred target sites of the hypothalamic-associated circuitry to affect specific hypothalamic-related conditions are listed in Table II.

TABLE I
Hypothalamic-Associated Circuitry

Hypothalamus; Limbic Structures; Olfactory Bulbs; Olfactory Nerves;
Hippocampus; Amygdala; Nucleus Solitarius; Reticular Formation;
Optic Nerve; Retina; Circumventricular Organs; Organum Vasculosum
Lamina Terminalis; Area Postrema; Lateral Medulla; Pituitary Gland;
Fornix; Septal Nuclei of Fornix; Stria Terminalis; Stria Medullaris;
Amygdalo-Hypothalamic Tract; Amygdalofugal Fibers; Medial
Forebrain Bundle; Pallidohypothalamic Tract; Cranial Nerve Nuclei;
Mammillothalamic Tract; Mammillotegmental Tract; Hypothalamo-
Hypophyseal System; Entorhinal Cortex; Pyriform Cortex; Septal
Region; Dorsal Longitudinal Fasciculus; Medial Parabrachial Nucleus;
Lateral Parabrachial Nucleus; Cingulate Gyrus; Ventral Noradrenergic
Bundle; Dorsal Noradrenergic Bundle; Locus Coeruleus; Vagal
Motor Nucleus; Nucleus Ambiguus; Ventrolateral Medulla;
TuberInfundibular Tract; Median eminence; Infundibulum; Midbrain
Raphe Nuclei; Periaqueductal Gray; Ventral Tegmental Region;
Dorsal/Midbrain Tegmental Nuclei; Nucleus of Diagonal Band;
Pineal Gland

TABLE II
Target Sites of Hypothalamic-Associated Circuitry for
Specific Hypothalamic-Related Conditions

| Target Site | Hypothalamic-Related Condition |
|---|---|
| Hypothalamus | Psychogenic Polydipsia; Raynaud's Syndrome; Infertility; Ovulation |
| | Irregular/painful Menses; Hypogonadism; Low Sperm Count; Precocious |
| | Puberty; Hyperthermia; Hypothermia; Lack of or Hyper-Lactation; |
| | Gastroparesis; Hypothyroidism; Hyperthyroidism; Pulmonary Edema; |
| | Prolactinoma; Syndrome of Inappropriate ADH Secretion (SIADH); |
| | Diabetes Insipidus; Acromegaly; Dwarfism; Rage Disorder; Hypertension; |
| | Orthostatic Hypotension; Taste Disorder; Arrhythmia/Bradycardia; |
| | Chronic pain; Heart Failure; Coma; Lethargy; Sexual Disorders; |
| | Hyperhidrosis; Narcolepsy; Epilepsy; Angina; Insomnia; Movement |
| | Disorders; Circadian Rhythms; Asthma; Facial Blushing; Autonomic |
| | Instability; Impotence; Baldness; Pain; Disorders of the eye, lacrimary and |
| | salivary glands, liver, heart, esophagus, lungs, stomach (gastrointestinal), |
| | pancreas, small intestine, large intestine, rectum, bladder, reproductive |
| | organs |
| Amygdala | Psychogenic Polydipsia; Taste Disorder; Rage Disorder; Sexual Disorders; |
| | Narcolepsy; Insomnia; Circadian Rhythms |
| Tuberoinfundibular Tract | Infertility; Ovulation; Irregular/Painful menses; Hypogonadism; Low |
| | Sperm Count; Precocious Puberty; Lack of or Hyper-Lactation; |
| | Hypothyroidism; Hyperthyroidism; Pulmonary Edema; Prolactinoma; |
| | SIADH; Diabetes Insipidus; Acromegaly; Dwarfism; Hypertension; |
| | Addictions; Coma; Lethargy; Obesity; Chronic pain; Anorexia; |
| | Psychogenic Polydipsia; Hypothermia |
| Locus Coeruleus | Pulmonary Edema; Rage Disorders; Hypertension; Heart Failure; |
| | Arrhythmia/Bradycardia; Obesity; Narcolepsy; Insomnia; Circadian |
| | Rhythm |
| Stria Terminalis | Psychogenic Polydipsia; Addiction; Fear/Anxiety; Sexual Disorders; |
| | Chronic pain; Insomnia; Narcolepsy; Circadian Rhythm |
| Dorsal Longitudinal | Rage Disorder; Chronic pain; Lethargy; Coma; Obesity; Addictions; |
| Fasciculus | Anorexia; Movement Disorders; Psychiatric Disorders; Insomnia; |
| | Narcolepsy; Circadian Rhythm |
| Nucleus Ambiguus | Raynaud's Syndrome; Gastroparesis; Hypertension; |
| | Arrhythmia/Bradycardia; Obesity |
| Nucleus Solitarius | Rage Disorders; Orthostatic Hypotension; Gastroparesis; Heart Failure; |
| | Angina; Hypertension |
| Ventral Noradrenergic | Gastroparesis |
| Bundle | |
| Medial Parabrachial Nucleus | Taste Disorders |
| Lateral Parabriachal Nucleus | Psychogenic Polydipsia; Raynaud's Syndrome; Hyperhidrosis; |
| | Gastroparesis |
| Vagal Motor Nucleus | Taste Disorders; Gastroparesis; Hypertension |
| Fornix | Obesity; Lethargy |
| Medial Forebrain Bundle | Orthostatic Hypotension; Heart Failure; Rage Disorder; Angina; |
| | Hypertension; Depression; Anxiety; Insomnia; Narcolepsy; Circadian |
| | Rhythm; Chronic pain |
| Periaqueductal Gray | Insomnia; Addictions; Obesity; Anorexia; Rage Disorder; Psychiatric |
| | Disorders |
| Ventral Tegmental Region | Hypothermia; Hypothermia |
| Pituitary Gland | Diabetes Insipidus; SIADH; Psychogenic Polydipsia; Irregular/Painful |
| | menses; Hypogonadism; Low Sperm Count; Precocious Puberty; |
| | Hypothermia; No or Hyper-Lactation; Hypothyroidism; Hyperthyroidism; |
| | Prolactinoma; Acromegaly; Dwarfism; Hypertension |
| Dorsal Noradrenergic Bundle | Rage; Depression |
| Dorsal/Midbrain Tegmental | Hypertension |
| Nuclei | |
| Ventrolateral Medulla | Arrhythmia/Bradycardia |
| Infundibulum | Infertility; Ovulation; Psychogenic Polydipsia; Irregular/Painful menses; |
| | Hypogonadism; Low Sperm Count; Precocious Puberty; Hypothermia; No |
| | or Hyper-Lactation; Hyperthyroidism; Hypothyroidism; Prolactinoma; |
| | SIADH; Acromegaly; Dwarfism; Hypertension; Diabetes Insipidus; Rage |
| | Disorders |
| Median Eminence | No or Hyper-Lactation; Psychogenic Polydipsia; Irregular/Painful menses; |
| | Hypogonadism; Low Sperm Count; Precocious Puberty; Hypothermia; |
| | Hypothyroidism; Hyperthyroidism; Prolactinoma; SIADH; Acromegaly; |
| | Dwarfism; Diabetes Insipidus |
| Septal Nuclei of Fornix | Heart Failure |
| Nucleus of Diagonal Band | Anxiety; Addictions |
| Amygdalofugal Fibers | Anxiety |
| Midbrain Raphe Nuclei | Narcolepsy; Insomnia; Circadian Rhythm |
| Retina/Optic Nerve | Circadian Rhythm; Narcolepsy; Insomnia |
| Pineal Gland | Insomnia; Narcolepsy; Circadian Rhythm |
| Stria Medullaris | Rage Disorders |

In another embodiment of the present invention, a method of affecting a hypothalamic-related disorder includes implanting a stimulator in a target site of a hypothalamus and providing a stimulation signal to the stimulator to stimulate the target site to affect the hypothalamic-related condition. The hypothalamus according to the present invention is a collection of nuclei and fibers in the lower part of the diencephalon of the brain, and unless otherwise distinguished, includes the pituitary gland. Referring to Table III, the hypothalamus includes the optic chiasm; median eminence; the infundibulum; mammillary bodies; anterior lobe of the pituitary gland; posterior lobe of the pituitary gland; and various tracts/fiber systems that project to, from, through, or within the hypothalamus. Although not wishing to be bound by theory, preferred target sites of the hypothalamus to affect specific hypothalamic-related conditions are listed in Table IV.

TABLE III
Hypothalamus

Hypothalamus; Median eminence; Infundibulum; Mammillary
Bodies; Anterior Lobe of Pituitary Gland; Posterior Lobe
of Pituitary Gland; Medial Forebrain Bundle; Fornix;
Mammillothalamic Tract; Stria Medullaris; Stria Terminalis;
Hypothalamo-Hypophyseal Portal; Tuberinfundibular Tract;
Dorsal Longitudinal Fasciculus

TABLE IV
Target Sites of Hypothalamus for Specific
Hypothalamic-Related Conditions

| Target Site | Hypothalamic-Related Condition |
|---|---|
| Hypothalamus | Psychogenic Polydipsia; Raynaud's Syndrome; Infertility; |
| | Ovulation; Irregular/Painful menses; Hypogonadism; Low Sperm |
| | Count; Precocious Puberty; Hyperthermia; Hypothermia; Lack of or |
| | Hyper-Lactation; Gastroparesis; Hypothyroidism; Hyperthyroidism; |
| | Pulmonary Edema; Prolactinoma; SIADH; Diabetes Insipidus; |
| | Acromegaly; Dwarfism; Rage Disorder; Hypertension; Orthostatic |
| | Hypotension; Taste Disorder; Arrhythmia/Bradycardia; Chronic |
| | pain; Heart Failure; Coma; Lethargy; Sexual Disorders; |
| | Hyperhidrosis; Narcolepsy; Epilepsy; Angina; Movement |
| | Disorders; Insomnia; Asthma; Facial Blushing; Autonomic |
| | Instability; Impotence; Baldness; Pain; Disorders of the eye, |
| | lacrimary and salivary glands, liver, heart, esophagus, lungs, |
| | stomach (gastrointestinal), pancreas, small intestine, large intestine, |
| | rectum, bladder, reproductive organs |
| Stria Medullaris | Rage Disorders |
| Stria Terminalis | Psychogenic Polydipsia; Addiction; Fear/Anxiety; Sexual |
| | Disorders; Chronic pain; Insomnia; Narcolepsy; Circadian Rhythm |
| Medial Forebrain Bundle | Orthostatic Hypotension; Heart Failure; Rage Disorder; Angina; |
| | Hypertension; Depression; Anxiety; Insomnia |
| | Insomnia; Narcolepsy; Circadian Rhythm; Chronic pain |
| Infundibulum | Infertility; Ovulation; Psychogenic Polydipsia; Hypogonadism; |
| | Low Sperm Count; Precocious Puberty; Hypothermia; No or |
| | Hyper-Lactation; Hyperthyroidism; Hypothyroidism; Prolactinoma; |
| | SIADH; Acromegaly; Dwarfism; Hypertension; Irregular/Painful |
| | menses; Diabetes Insipidus; Rage Disorders |
| Median Eminence | No or Hyper-Lactation; Irregular/Painful menses; Hypogonadism; |
| | Precocious Puberty; Hypothermia; Hyperthyroidism; |
| | Hyperthyroidism; Prolactinoma; SIADH; Acromegaly; Dwarfism; |
| | Psychogenic Polydipsia; Low Sperm Count; Diabetes Insipidus |
| Tuberoinfundibular Tract | Infertility; Ovulation; Irregular/Painful menses; Hypogonadism; |
| | Low Sperm Count; Precocious Puberty; Lack of or Hyper- |
| | Lactation; Hypothyroidism; Hyperthyroidism; Pulmonary Edema; |
| | Prolactinoma; SIADH; Diabetes Insipidus; Acromegaly; Dwarfism; |
| | Hypertension; Addictions; Coma; Lethargy; Obesity; Chronic pain; |
| | Anorexia; Psychogenic Polydipsia; Hypothermia |
| Dorsal Longitudinal Fasciculus | Rage Disorder; Chronic pain; Lethargy; Coma; Obesity; |
| | Addictions; Anorexia; Movement Disorders; Psychiatric Disorders; |
| | Insomnia; Narcolepsy; Circadian Rhythm |
| Fornix | Obesity; Coma; Lethargy |
| Anterior Lobe of Pituitary | Psychogenic Polydipsia; Irregular/Painful menses; Hypogonadism; |
| | Low Sperm Count; Precocious Puberty; Hypothermia; No or |
| | Hyper-Lactation; Hyperthyroidism; Hypothyroidism; Prolactinoma; |
| | Acromegaly; Dwarfism; Hypertension |
| Posterior Lobe of Pituitary | SIADH; Psychogenic Polydipsia; No or Hyper-Lactation |
| Mammillary Body | Gastroparesis; Hypertension; Angina |

In another embodiment of the present invention, a method of treating a hypothalamic-related disorder includes implanting a stimulator in a target site of a division of the hypothalamus and providing a stimulation signal to the stimulator to stimulate the target site to affect the hypothalamic-related condition. Referring to Table V, the divisions of the hypothalamus according to the present invention are the periventricular, medial, and lateral divisions. Although not wishing to be bound by theory, preferred target sites of the divisions of the hypothalamus to affect specific hypothalamic-related conditions are listed in Table VI.

TABLE V

| Division of | Components of |
|---|---|
| Hypothalamus | Division of Hypothalamus |
| Periventricular | Arcuate Nucleus; Paraventricular |
| | Nucleus/Periventricular Nucleus; Suprachiasmatic |
| | Nucleus; Tuberoinfundibular Tract; Stria Terminalis; |
| | Dorsal Longitudinal Fasciculus; Fornix; Medial |
| | Forebrain Bundle; Optic Chiasm |
| Medial | Anterior Nucleus; Supraoptic Nucleus; |
| | Preoptic Nucleus; Mammillary |
| | Body; Ventromedial Nucleus; Dorsomedial Nucleus; |
| | Posterior Nucleus; Substantia Innominata; Basal |
| | Nucleus of Meynert; Stria Terminalis; Tuberinfundibular |
| | Tract; Dorsal Longitudinal Fasciculus; Medial |
| | Forebrain Bundle; Fornix; Mammillothalamic Tract; |
| | Mammilotegmental Tract; Mammillary Peduncle; |
| | Pallidohypothalamic Tract |
| Lateral | Preoptic Nucleus; Supraoptic Nucleus; |
| | Lateral Nucleus; Tuberal Nuclei; |
| | Medial Forebrain Bundle, Fornix; Amygdalofugal |
| | fiber; Stria Terminalis; Median eminence, |
| | Tuberoinfundibular Tract; and Dorsal |
| | Longitudinal Fasciculus |

TABLE VI
Target Divisions of Hypothalamus for Specific Hypothalamic-Related Conditions

| Division of | |
|---|---|
| Hypothalamus | Hypothalamic-Related Condition |
| Periventricular | Chronic pain; Gastroparesis; Orthostatic Hypotension; |
| | Arrythymia/Bradiacardia; Heart Failure; Hypertension; Pulmonary Edema; |
| | Prolactinoma; SIADH; Diabetes Insipidus; Lethargy; Coma; Acromegaly; |
| | Dwarfism; Infertility; Ovulation; Irregular/Painful menses; Hypogonadism; |
| | Low Sperm Count; Precocious Puberty; Lack of or Hyper-Lactation; |
| | Hypothyroidism; Hyperthyroidism; Addictions; Raynaud's Syndrome; |
| | Hyperthermia; Fear; Anxiety; Sexual Disorders; Rage Disorder; Anorexia; |
| | Epilepsy; Hypothermia; Rage Disorder; Angina; Depression; Insomnia; |
| | Hypothermia; Hyperhidrosis; Narcolepsy; Circadian Rhythms; Psychogenic |
| | Polydipsia, Asthma |
| Medial | Addiction; Sexual Disorders; Hyperhidrosis; Hyperthermia; Raynaud's |
| | Syndrome; Infertility; Ovulation; Irregular/Painful menses; Hypogonadism; |
| | Low Sperm Count; Precocious Puberty; Lack of or Hyper-Lactation; |
| | SIADH, Diabetes Insipidus; Gastroparesis; Hypertension; Angina; |
| | Arrhythmia/Bradycardia; Raynaud's Syndrome; Rage Disorder; Depression; |
| | Epilepsy; Movement Disorders; Taste Disorders; Heart Failure; |
| | Hypothermia; Insomnia; Narcolepsy; Hypothyroidism; Hyperthyroidism; |
| | Lethargy; Acromegaly; Dwarfism; Orthostatic Hypotension; Chronic Pain; |
| | Asthma |
| Lateral | Rage; Depression; Anxiety; Epilepsy; Addiction; Chronic pain; Infertility; |
| | Ovulation; Irregular/Painful menses; Hypogonadism; Low Sperm Count; |
| | Precocious Puberty; Gastroparesis; Hypertension; Angina; |
| | Arrythymia/Bradycardia; Raynaud's Syndrome; Lack of or Hyper-Lactation; |
| | SIADH; Diabetes Insipidus; Hypothermia; Orthostatic Hypotension; |
| | Acromegaly; Dwarfism; Heart Failure; Rage Disorder; Depression; |
| | Insomnia; Lethargy; Coma; Hyperhidrosis; Sexual Disorders; Psychogenic |
| | Polydipsia; Hypothyroidism; Hyperthyroidism; Pulmonary Edema; Chronic |
| | Pain; Asthma; Autonomic Instability |

In another embodiment of the present invention, a method of treating a hypothalamic-related disorder includes implanting a stimulator in a nucleus of the hypothalamus and providing a stimulation signal to the stimulator to stimulate a nucleus of the hypothalamus to affect the hypothalamic-related condition. Although there are may nuclei in the hypothalamus and the present invention contemplates implanting a stimulator in any one of these nuclei, referring to Table VII, in a preferred embodiment of the present invention, a stimulator is implanted in the anterior nucleus, arcuate nucleus, paraventricular nucleus, supraoptic nucleus, preoptic nucleus, lateral nucleus, tuberal nucleus, suprachiasmatic nucleus, mammillary body, ventromedial nucleus, dorsomedial nucleus, poster nucleus, substantia innominate, or basal nucleus of meynert. Although not wishing to be bound by theory, preferred nuclei of the hypothalamus to affect specific hypothalamic-related conditions are listed in Table VIII.

TABLE VII
Nuclei of Hypothalamus

Anterior Nucleus; Arcuate Nucleus; Paraventricular Nucleus/
Periventricular Nucleus; Supraoptic Nucleus; Preoptic Nucleus;
Lateral Nucleus, including tuberal nucleus; Suprachiasmatic
Nucleus; Mammillary body; Ventromedial Nucleus; Dorsomedial
Nucleus; Posterior Nucleus; Substantia Innominata; Basal
Nucleus of Meynert

TABLE VIII
Target Nuclei for Specific Hypothalamic-Related Conditions

| Nucleus of Hypothalamus | Hypothalamic-Related Condition |
|---|---|
| Anterior Nucleus | Addiction; Fear/Anxiety; Sexual Disorders; Hyperhidrosis; |
| | Hyperthermia; Psychogenic Polydipsia; Raynaud's Syndrome; |
| | Infertility; Ovulation; Irregular/Painful menses; Hypogonadism; Low |
| | Sperm Count; Precocious Puberty; Impotence; Baldness |
| Arcuate Nucleus | No or Hyper-Lactation; Chronic pain; Infertility; Ovulation; |
| | Irregular/Painful menses; Hypogonadism; Low Sperm Count; |
| | Precocious Puberty; Impotence; Baldness |
| Periventricular | Chronic pain; Gastroparesis; Orthostatic Hypertension; |
| Nucleus/ | Arrythymia/Bradiacardia; Heart Failure; Hypo/Hyperthyroidism; |
| Paraventricular | Hypertension; Pulmonary Edema; No or Hyper-Lactation; |
| Nucleus | Prolactinoma; SIADH; Diabetes Insipidus; Lethargy; Coma; |
| | Acromegaly; Dwarfism |
| Supraoptic Nucleus | Addictions; No or Hyper-Lactation; SIADH; Diabetes Insipidus; |
| | Gastroparesis; Hypertension; Angina; Arrhythmia/Bradycardia; |
| | Raynaud's Syndrome; Impotence |
| Preoptic Nucleus | Rage; Depression, Anxiety, Epilepsy; Addiction; Chronic pain; |
| | Infertility; Ovulation; Irregular/Painful menses; Hypogonadism; Low |
| | Sperm Count; Precocious Puberty; Gastroparesis; Hypertension; |
| | Angina; Arrythymia/Bradycardia; Raynaud's Syndrome; Impotence; |
| | Baldness |
| Lateral Nucleus including the | Rage Disorder; Epilepsy; Anxiety; Addictions; Obsessive/Compulsive |
| Tuberal Nucleus | Disorders; Heart Failure; Hypothermia; Insomnia; Narcolepsy; |
| | Gastroparesis; Hypertension; Angina; Arrhythmia/Bradycardia; |
| | Raynaud's Syndrome; Hyperhidrosis; Hyperthermia; Psychogenic |
| | Polydipsia |
| Suprachiasmatic Nucleus | Circadian Rhythms; Insomnia; Narcolepsy |
| Mammillary Body | Epilepsy; Chronic pain; Gasteroparesis; Hypertension; Angina |
| Ventromedial Nucleus | Chronic pain; Addictions; Rage Disorder; Movement Disorders; |
| | Psychiatric Disorders; Infertility; Ovulation; Irregular/Painful menses; |
| | Hypogonadism; Low Sperm Count; Precocious Puberty; Impotence; |
| | Baldness |
| Dorsomedial Nucleus | Epilepsy; Chronic pain; Depression; Rage; Hypo/Hyperthyroidism |
| Posterior Nucleus | Chronic pain; Taste Disorder; Rage Disorder; Hypertension; Anxiety; |
| | Heart Failure; Hypothermia; Insomnia; Narcolepsy; Precocious |
| | Puberty; Hypogonadism |
| Substantia Innominata | Taste Disorders; Chronic pain; Depression; Anxiety |
| Basal Nucleus of Meynart | Alzheimer's Disease; Dementias |

The foregoing conditions and corresponding target sites are not exclusive both for the listed conditions and other hypothalamic-related conditions and other target sites can be identified from anatomical studies, animal studies, autopsies, imaging techniques such as magnetic resonance imaging (MRI) and computerized tomography (CT) scans, electroencephalography (EEG), magnetoencephalography (MEG), metabolic and blood flow techniques such as functional magnetic resonance imaging (fMRI), positron emission tomography (PET), and other physiological and diagnostic tools to understand normal functioning or activity of the brain and the abnormal functioning manifested in hypothalamic-related conditions. One particular method envisioned by the present invention to identify the proper target sites for a specific hypothalamic-related condition includes identifying a large sampling of patients who each exhibit symptoms of the specific hypothalamic-related condition and identifying which common regions of the brain exhibit pathological electrical and/or chemical activity during manifestation of the specific hypothalamic-related condition. The common regions demonstrating this pathological activity constitute the target site and a stimulator may then be implanted in or in communication with this target site of a patient. Further details of methods of determining the proper therapeutic target site by identifying common pathological regions in a large sampling of patients is described in U.S. Pat. Nos. 6,167,311 and 6,418,344, both of which are incorporated by reference herein.

Although the common region demonstrating pathological activity generally constitutes the target site in a patient, the exact location of the target site may vary from patient to patient. Accordingly, as described in greater detail below, standard neurological procedures can be used to localize the x, y, and z coordinates of the target site in a specific patient. For example, a CT scan, an MRI scan, and computerized standard brain atlas can be used to create a 3-dimensional image of a patient's brain and within that image the x, y, and z, coordinates can be identified. In addition, as will be readily recognized by one of skill in the art, stereotactic coordinates for any of the hypothalamic-related target sites according to the present invention can be determined from a number of brain atlases such as Schaltenbrand, G and Bailey P, Introduction to Stereotaxis, with an Atlas of the Human Brain (1959, G. Thieme, Stuttgart); Schaltenbrand, G. and Wahren, W. Atlas for Stereotaxy of the Human Brain (1977, Thieme, Stuttgart); J. Mai, J. Assheuer, and George Paxinos, Atlas of the Human Brain (1997 Academic Press), all of which are incorporated by reference herein.

In another embodiment of the present invention, a method of treating a hypothalamic-related condition includes implanting a stimulator in a target site of the hypothalamus and/or pituitary gland, providing a stimulation signal to the stimulator to stimulate the target site to modulate the synthesis or release of a hypothalamic or pituitaric product by inhibiting, blocking or stimulating the synthesis or release of such hormone. Referring to Table IX, hypothalamic products are substances synthesized and released by the hypothalamus including hormones and neuropeptides and pituitary products are hormones released by the pituitary including trophic hormones, the release of which are either inhibited or stimulated by hypothalamic hormones. Although not wishing to be bound by theory, specific products to be modulated to affect specific hypothalamic-related conditions are listed in Table X.

TABLE IX
Hypothalamic and Pituitary Products

| Products Synthesized | Correspondingly Regulated |
|---|---|
| or Released by | Product Released by |
| Hypothalamus | the Pituitary |
| Growth Hormone Releasing | Growth Hormone (GH) |
| Hormone (GHRH) | |
| Growth Hormone Release-Inhibiting | GH |
| Hormone (Somatostatin) | Thyrotropin (TSH) |
| Thyrotropin-Releasing Hormone (TRH) | TSH |
| | Prolactin (PRL) |
| Prolactin Releasing Hormone (PRH) | PRL |
| Prolactin-Inhibiting Hormone | |
| (PIH) (Dopamine) | |
| Gonadotropin-Releasing | Follicle Stimulating |
| Hormone (GnRH) | Hormone (FSH) |
| | Lutenizing Hormone (LH) |
| Corticotrophin-Releasing | Adrenocorticotropic |
| Hormone (CRH) | Hormone (ACTH) |
| | Betalipotropin; Melanocyte |
| | Stimulating Hormone |
| | (MSH); Beta endorphin |
| Antiduretic Hormone | ADH |
| (ADH) (Vasopressin) | |
| Vasopressin | |
| Oxytocin | |
| Glucogen | |
| Cholecystokinin | |
| Dynorphin | |
| Angiotensin | |
| Beta-Endorphin | |
| Enkaphalin | |

TABLE X
Target Hypothalamic or Pituitary Products to be
Modulated for Specific Hypothalamic-Related Conditions

| | Correspondingly Affected |
|---|---|
| Hypothalamic or | Hypothalamic-Related |
| Pituitary Products | Conditions |
| GHRH, GH | Acromegaly |
| Somatostatin | Acromegaly, |
| Vasopression | Congestive Heart Failure |
| Oxytocin, PRL, TRH, | Lack of Lactation |
| PRH, ACTH, | |
| Beta-Lipotrophin, | |
| CRH | |
| Oxytocin, PRL, PRH, TRH | Hyperlactation |
| Dopamine | Hyperlactation |
| Dopamine | Lack of Lactation |
| GnRH, FSH | Low Sperm Count; Hypogonadism |
| GnRH, LH, FSH | Infertility; Ovulation |
| TRH, TSH | Hypothyroidism |
| TRH, TSH | Hyperthyroidism |
| GnRH | Anorexia; Impotence; Baldness |
| Somatostatin | Hyperthyroidism |
| Somatostatin | Hypothyroidism |
| CRH; ACTH | Cushing's Disease |
| ADH | Diabetes Insipidus; Psychogenic Polydipsia |
| CRH; ACTH | Lethargy |
| GHRH; GH | Dwarfism |
| Somatostatin | Dwarfism |
| GnRH; LH | Precocious Puberty |
| LH | Irregular Menses |
| Glucogen | Diabetes; Angina |
| Cholecystokinin | Hypotension; Hypertension |
| Dynorphin | Arrhythmia/Bradycardia |
| Angiotensin | Heart failure; Raynaud's Syndrome |
| Beta-Endorphin | Lethargy; Pain; Narcolepsy; |
| | Anxiety; Depression; Addictions |
| Enkaphalin | Gastroparesis |

The stimulator may be implanted in either the hypothalamus or the pituitary gland. In embodiments where the stimulator is implanted in the hypothalamus, releasing and inhibiting hormones that are released by the hypothalamus and that regulate the release of pituitary hormones are directly modulated and pituitary hormones are indirectly modulated. Preferred target sites of the hypothalamus in which to perform such modulation are listed in Table XI. In embodiments where the stimulator is placed in the pituitary, hormones released by the pituitary are directly modulated and hormones released by the hypothalamus are indirectly modulated as a result of the negative feedback exerted upon the hypothalamus by the pituitary. Therefore, the decision of whether to implant the stimulator in the hypothalamus or the pituitary gland can be made, in part, on the desired level of regulation.

TABLE XI
Target Sites of Hypothalamus for Modulation of
Specific Hormones

| Target Site of Hypothalamus | Hormone Modulated |
|---|---|
| Anterior Nucleus | LH; GnRH |
| Arcuate Nucleus | Dopamine; ACTH; Betalipotrophin; |
| | Beta-endorphin; CRH |
| Paraventricular/ | TSH; Dopamine; Oxytocin; Vasopressin; |
| Periventricular Nucleus | CRH; Beta-endorphin; Somatostatin |
| Supraoptic Nucleus | GH; Oxytocin; Vasopressin; GHRH |
| Preoptic Nucleus | LH; GnRH |
| Ventromedial Nucleus | LH; GnRH |
| Dorsomedial Nucleus | TSH; TRH |
| Posterior Nucleus | LH; GnRH |

Although this embodiment of the present invention contemplates electrical and/or chemical stimulation to affect the synthesis or release of a hypothalamic or pituitary product, this embodiment is particularly useful for chemical stimulation as chemical agents can be delivered directly to the hypothalamus or pituitary. Such chemical agents include antagonists, agonists, other therapeutic neuromodulation agents, and combinations thereof that bind to the receptors of hypothalamic and pituitary products to regulate the actions thereof. Although such chemical agents are generally administered orally in traditional pharmacotherapies, by directly stimulating the target sites in the brain that synthesize or release such products, low and precise doses of the chemical agents can be administered so as to minimize or avoid the side effects and delayed onset of relief common to traditional pharmacotherapy.

With respect to particular details of chemical stimulation according to the present invention, whether employed alone or in combination with electrical stimulation, once the stimulator (i.e. a catheter) is secured in place in the hypothalamic-related target site, the stimulation controller (i.e. drug pump) is activated thereby delivering a chemical agent to the target site. The chemical agent may be a neurotransmitter mimick; neuropeptide; hormone; pro-hormone; antagonist, agonist, reuptake inhibitor, or degrading enzyme thereof; peptide; protein; therapeutic agent; amino acid; nucleic acid; or stem cell and may be delivered by a slow release matrix or drug pump. The chemical agents may also be delivered continuously or intermittently.

With respect to particular details of electrical stimulation according to the present invention, once the stimulator (i.e. electrode) is secured in place in the hypothalamic-related target site, the stimulation controller (i.e. pulse generator) is activated thereby applying to the target site an oscillating electrical signal having specified pulsing parameters. The oscillating electrical signal may be applied continuously or intermittently and the pulsing parameters, such as the pulse width, amplitude, frequency, voltage, current, intensity, pole combinations, and/or waveform may be adjusted to affect a desired result. Preferably, the oscillating electrical signal is operated at a voltage between about 0.1 μL V to about 20 V. More preferably, the oscillating electrical signal is operated at a voltage between about 1 V to about 15 V. Preferably, the electric signal is operated at a frequency range between about 2 Hz to about 2500 Hz. More preferably, the electric signal is operated at a frequency range between about 2 Hz to about 200 Hz. Preferably, the pulse width of the oscillating electrical signal is between about 10 microseconds to about 1,000 microseconds. More preferably, the pulse width of the oscillating electrical signal is between about 50 microseconds to about 500 microseconds. The waveform may be, for example, biphasic square wave, sine wave, or other electrically safe and feasible combination. Preferably, the application of the oscillating electrical signal is: monopolar when the electrode is monopolar, bipolar when the electrode is bipolar, and multipolar when the electrode is multipolar.

Although certain embodiments of the present invention are particularly advantageous for either chemical or electrical stimulation, the present invention contemplates either type of stimulation or both types of stimulation of a hypothalamic-related target site to affect a hypothalamic-related condition. One non-limiting example of the use of chemical and electrical stimulation to affect a hypothalamic-related condition, particularly one characterized by cellular damage at the target site involves repopulating the target site with undifferentiated cells or nucleic acids and stimulating the growth of such cells or replication of such nucleic acids by electrical stimulation. Such repopulation of cells can be carried out using a cellular or molecular approach. Cellular approaches involve injecting or infusing undifferentiated cells, which are preferably cultured autologous cells, into the target site. Molecular approaches involve injecting or infusing nucleic acids, whether in the form of naked or plasmid DNA, into the target site. Methods of delivering nucleic acids to a cellular target site are well known in the art and generally involve the use of delivery vehicles such as expression vector or liposomes. Non-limiting examples of expression vectors for use in this embodiment of the present invention include bacterial expression vectors and viral expression vectors such as retroviruses, adenoviruses, or adeno-associated viral vectors.

In the case of repopulating the target site with nucleic acid molecules, such molecules are preferably recombinant nucleic acid molecules and can be prepared synthetically or, preferably, from isolated nucleic acid molecules, as is known in the art. A nucleic acid is “isolated” when it is purified away from other cellular constituents, such as, for example, other cellular nucleic acids or proteins by standard techniques known to those of skill in the art. The coding region of the nucleic acid molecule can encode a full length gene product or a fragment thereof or a novel mutated or fusion sequence. The coding sequence can be a sequence endogenous to the target cell, or exogenous to the target cell. The promoter, with which the coding sequence is operably associated, may or may not be one that normally is associated with the coding sequence.

The cellular or genetic material can be delivered simultaneously with the electrical stimulation, or the cellular or genetic material can be delivered separately. One particularly advantageous feature of this embodiment of combined chemical and electrical stimulation is that the expression of the nucleic acid molecules may be regulated by electrical stimulation. Namely, the amplitude, intensity, frequency, duration and other pulsing parameters may be used to selectively control expression of nucleic acid molecules delivered to the target site. Further details of the use of electrical stimulation and nucleic acid delivery to repopulate a target site are described in U.S. Pat. No. 6,151,525, which describes the use of electrical current to modify contractile cells to form new contractile tissue and which is incorporated by reference herein.

Another example of electrical and chemical stimulation being used together, is the use of electrical stimulation to modulate the expression of cellular receptors at the target site.

Notwithstanding whether chemical and/or electrical stimulation is employed in the methods of the present invention, the present invention also contemplates the use of a closed-loop feedback mechanism in conjunction with chemical or electrical stimulation. In such an embodiment, a hypothalamic-related target site is stimulated in response to a physiological sensor signal. In particular, this embodiment includes implanting a stimulator in communication with a hypothalamic-related target site, detecting a physiological activity of the body associated with the hypothalamic-related condition to generate a sensor signal and providing a stimulation signal to a stimulator in response to the sensor signal to stimulate the target site and affect the hypothalamic-related condition. Such physiological activity to be detected is a physiological characteristic or function of the body, and includes, for example, body temperature regulation, blood pressure, metabolic activity, cerebral blood flow, pH levels, vital signs, galvanic skin responses, electrocardiogram, electroencephalogram, action potential conduction, and hormone, electrolyte, glucose or other chemical production.

In particular, the physiological activity of the body may include any electrical or chemical activity of the body and may be detected by sensors located on or within the body. For example, such activity may be detected by sensors located within or proximal to the target site, distal to the target site but within the nervous system, or by sensors located distal to the target site outside the nervous system. Examples of electrical activity detected by sensors located within or proximal to the target site include sensors that measure neuronal electrical activity, such as the electrical activity characteristic of the signaling stages of neurons (i.e. synaptic potentials, trigger actions, action potentials, and neurotransmitter release) at the target site and by afferent and efferent pathways and sources that project to and from or communicate with the target site. For example, if the hypothalamic-related condition being affected is hypertension and the target site is the preoptic nucleus, then sensors can measure, at any signaling stage, neuronal activity of the preoptic nucleus, the medial forebrain bundle, and the vagal motor nucleus. In particular, the sensors may detect the rate and pattern of the neuronal electrical activity to determine the stimulation signal to be provided to the stimulator.

Examples of chemical activity detected by sensors located within or proximal to the target site include sensors that measure neuronal activity, such as the modulation of neurotransmitters, hormones, pro-hormones, neuropeptides, peptides, proteins, electrolytes, or small molecules by the target site and modulation of these substances by afferent and efferent pathways and sources that project to and from the target site or communicate with the target site. Local levels of these substances may be measured using in vivo microdialysis, which provides a measure of the levels of these substances in the intercellular fluid. For example, if the hypothalamic-related condition being treated is lack of lactation and the target site is the supraoptic nucleus, then sensors can measure, at any stage, the presence and/or amount of oxytocin released by the supraoptic nucleus into the hypophyseal portal veins to the posterior pituitary.

With respect to detecting electrical or chemical activity of the body by sensors located distal to the target site but still within the nervous system, such sensors could be placed in the brain, the spinal cord, cranial nerves, and/or spinal nerves. Sensors placed in the brain are preferably placed in a layer-wise manner in the direction of increasing proximity to the target site. For example, a sensor could be placed on the scalp (i.e. electroencephalogram), in the subgaleal layer, on the skull, in the dura mater, in the sub dural layer and in the parenchyma (i.e. in the frontal lobe, occipital lobe, parietal lobe, temporal lobe) to achieve increasing specificity of electrical and chemical activity detection. The sensors could measure the same types of chemical and electrical activity as the sensors placed within or proximal to the target site as described above.

With respect to detecting electrical or chemical activity by sensors located distal to the target site outside the nervous system, such sensors may be placed in venous structures and various organs or tissues of other body systems, such as the endocrine system, circulatory system, urinary system, integumentary system, and digestive system or such sensors may detect signals from these various body systems depending on the particular hypothalamic-related condition desired to be affected and the corresponding physiological activity desired to be measured. For example, if the hypothalamic-related condition being affected is gastropareisis, then sensors may be placed in the stomach to detect electrical and muscular activity, pH level, and gastric content of enzymes and hormones of the stomach. If the hypothalamic-related condition being affected is hyperhidrosis then sensors may be placed on the skin to detect perspiration. If the hypothalamic-related condition is arrythymias, bradycardia, or angina, then sensors may be placed on the skin to measure electrocardiograms. If the hypothalamic-related disorder is prolactinoma, hyperthyroidism, psychogenic polydipsia, diabetes insipidus, or another disorder related to hormonal, electrolyte or other chemical imbalance, sensors may be placed in contact with the bladder to measure hormonal, electrolyte, glucose levels, or other chemical levels in the urine. Sensors may also be placed in venous structures such as the jugular vein to measure these same chemical substances in the blood. The above-mentioned physiological activities and the corresponding locations of sensors are merely exemplary. Other specific physiological activities and locations on or in the body to place sensors will be readily known to one of skill in the art for both the above-mentioned conditions as well as other hypothalamic-related conditions based on the symptoms, attributes and/or pathophysiology of such conditions. Furthermore, all the above-mentioned sensing systems may be employed together or any combination of less than all sensors may be employed together.

After the sensor(s) detect the relevant physiological activity associated with the targeted hypothalamic-related condition, the sensors generate a sensor signal. The sensor signal is processed by a sensor signal processor and provides a control signal to the stimulation controller, which is a signal generator or drug pump depending on whether electrical or chemical stimulation is desired. The stimulation controller, in turn, generates a response to the control signal by providing a stimulation signal to the stimulator. The stimulator then stimulates the target site to affect the hypothalamic-related condition. In the case of electrical stimulation, the control signal may be an indication to initiate, terminate, increase, decrease or to change the pattern or rate of a pulsing parameter of the electrical stimulation and the stimulation signal can be the respective initiation, termination, increase, or decrease or change in pattern or rate in the respective pulsing parameter. In the case of chemical stimulation, the control signal can be an indication to initiate, terminate, increase, decrease, or change the rate or pattern of the amount or type of chemical agent administered, and the stimulation signal can be the respective initiation, termination, increase, decrease, or change in the rate or pattern in the amount or type of chemical agent administered. The processing of closed-loop feedback systems for electrical and chemical stimulation are described in more detail in respective U.S. Pat. Nos. 6,058,331 and 5,711,316, both of which are incorporated by reference herein.

Although not wishing to be bound by the description of a particular procedure, one exemplary procedure effectuating the methods of the present invention shall now be described. Generally, the procedure begins with the patient having a stereotactic head frame mounted to the patient's skull, although frameless techniques may also be used. The patient then typically undergoes a series of MRI and/or CT sessions, during which a series of two dimensional slice images of the patient's brain are built up into a quasi-three dimensional map in virtual space. This map is then correlated to the three dimensional stereotactic frame of reference in the actual surgical field. In order to align these two coordinate frames, both the instruments and the patient must be situated in correspondence to the virtual map. A current method of achieving this alignment is to rigidly mount to the head frame to the surgical table. Subsequently, a series of reference points are established relative to aspects of the frame and patient's skull, so that the computer can adjust and calculate the correlation between the actual surgical field of the patient's head and the virtual space model of the patient's brain MRI scans. The surgeon is able to target any region within the stereotactic space of the brain with precision (e.g. within 1 mm). Initial anatomical localization of the hypothalamic-related target site is achieved either directly using the MRI images, or indirectly using interactive anatomical atlas programs that map the atlas image onto the stereotactic image of the brain. This indirect targeting approach involves entering the stereotactic anterior commissure (AC) and posterior commissure (PC) coordinates into a computer with a commercially available program containing digitized diagrams of sagittal brain sections from a standardized brain atlas. The program transcribes the patient's calculated AC-PC intercommissural line onto the digitized map at the sagittal laterality of interest. One these maps, the hypothalamic-related targets sites can be localized.

Another method of localizing the hypothalamic-related target site involves the fusion of functional and structural medical imaging. Such methods for localizing targets in the body and guiding diagnostic or therapeutic instruments toward a target region in the body have been described in U.S. Pat. No. 6,368,331, issued on Apr. 9, 2002 to Front et al., U.S. Patent Application Publication No. US 2002/0032375, published Mar. 14, 2002 by Bauch et al., and U.S. Patent Application Publication No. US 2002/0183607, published Dec. 5, 2002 by Bauch et al., all of which are hereby incorporated by reference in their entireties. Methods for target localization specifically within the nervous system, including the brain, have been described in U.S. Provisional Application No. 60/353,695, filed Feb. 1, 2002, by Rezai et al. which is hereby incorporated by reference in its entirety. Specifically, provided in U.S. Provisional Application No. 60/353,695 is a method of medical imaging, comprising: placing a fiducial marker proximate to an area of a body to be imaged; obtaining a first image of the area of the body using a first medical imaging technique, the first image including a first image of the fiducial marker; obtaining a second image of the area of the body using a second medical imaging technique, the second image including a second image of the fiducial marker, the second medical imaging technique being different than the first medical imaging technique; superimposing the first image of the area of the body and the second image of the area of the body; and aligning the first image of the first fiducial marker with the second image of the fiducial marker. Useful medical imaging techniques to obtain functional images include but are not limited to functional MRI, PET or MEG. Useful medical imaging techniques to obtain structural images include but are not limited to volumetric MRI, CT.

Subsequent to the stereotactic CT/MRI acquisition and anatomical localization, the patient is taken to the operating room. The surgery can be performed under either local or general anesthetic, but preferably under local anesthesia in order to allow communication with the patient. An initial incision is made in the scalp, preferably 2.5 centimeters lateral to the midline of the skull, anterior to the coronal suture. A burr hole is then drilled in the skull itself; the size of the hole being suitable to permit surgical manipulation and implantation of an electrode or catheter device. This size of the hole is generally about 14 millimeters. The dura is then opened, and fibrin glue is applied to minimize cerebral spinal fluid leaks and the entry of air into the cranial cavity. A guide tube cannula with a blunt tip is then inserted into the brain parenchyma to a point approximately one centimeter from the target tissue. At this time physiological localization starts with the ultimate aim of correlating the anatomical and physiological findings to establish the final stereotactic target structure.

Physiological localization using single-cell microelectrode recording is preferable performed for definitively identifying the hypothalamic-related target site by neuronal firing patterns of individual neurons. In addition to microelectrode recording, microstimulation and or macrostimulation may be performed to provide further physiological localization.

Once the final hypothalamic-related target site has been identified in the actual spatial frame of reference, the electrode is inserted into the target site and a hand-held pulse generator (Screener) is used for intraoperative test stimulation. Various pole combinations and stimulation frequency, pulse width, and intensity are used to determine the thresholds for therapeutic and adverse effects. Thereafter the electrode is locked into the burr hold ring to prevent lead migration. The proximal portion of the electrode is then attached to a transcutaneous pacing wire for a test trial period. After the test period, the patient undergoes implantation of a pulse generator or radio-frequency-coupled receiver.

Implanting the pulse generator is generally carried out with the patient under general anesthesia. The pulse generator is implanted in the infraclavicular space by tunneling from the frontal inicision to the infraclavicular space. The pulse generator can be powered by a battery and can be activated externally by an external transmitter.

Although the invention has been described with reference to the preferred embodiments, it will be apparent to one skilled in the art that variations and modifications are contemplated within the spirit and scope of the invention. The figures, tables, and description of the preferred embodiments are made by way of example rather than to limit the scope of the invention, and it is intended to cover within the spirit and scope of the invention all such changes and modifications.

1. A method of affecting a hypothalamic-related condition comprising:
implanting a stimulator in a target site of a hypothalamic-associated circuitry; and
providing a stimulation signal to the stimulator to stimulate the target site to affect the hypothalamic-related condition, wherein the hypothalamic-related condition is not an eating disorder.

2. The method of claim 1, wherein the target site is selected from the group consisting of the hypothalamus, stria terminalis, amygdala, hippocampus, amygdalofugal fibers, lateral parabrachial nucleus, nucleus ambiguus, locus coeruleus, tuberoinfundibular tract, medial parabrachial nucleus, ventral tegmental region, medial forebrain bundle, median eminence, vagal motor nucleus, nucleus solitarius, ventral noradrenergic bundle, dorsal noradrenergic bundle, fomix, septal nuclei of fomix, nucleus of diagonal band, mammillothalamic tract, dorsal longitudinal fasciculus, ventrolateral medulla, pituitary gland, midbrain raphe nuclei, periaqueductal gray, dorsal tegmental nuclei and pineal gland.

3. The method of claim 1, wherein the stimulator is an electrode.

4. The method of claim 1, wherein the stimulator is a catheter.

5. The method of claim 1, wherein the hypothalamic-related condition is selected from the group consisting of infertility, ovulation, irregular or painful menses, lack of lactation, hyperlactation, hypogonadism, low sperm count and precocious puberty.

6. The method of claim 1, wherein the hypothalamic-related condition is selected from the group consisting of psychogenic polydipsia, diabetes insipidus, and SIADH.

7. The method of claim 1, wherein the hypothalamic-related condition is selected from the group consisting of heart failure, arrhythmia, angina, hypertension, orthostatic hypotension, pulmonary edema, and asthma.

8. The method of claim 1, wherein the hypothalamic-related condition is gastroparesis or autonomic instability.

9. The method of claim 1, wherein the hypothalamic-related condition is acromegaly or dwarfism.

10. The method of claim 1, wherein the hypothalamic-related condition is selected from the group consisting of Raynaud's Syndrome, hyperhidrosis, hypothermia, hyperthermia and facial blushing.

11. The method of claim 1, wherein the hypothalamic-related condition is selected from the group consisting of hypothyroidism, hyperthyroidism, and prolactinoma.

12. The method of claim 1, wherein the hypothalamic-related condition is selected from the group consisting of insomnia, narcolepsy, lethargy, and circadian rhythms.

13. The method of claim 1, wherein the hypothalamic-related condition is sexual disorders or rage disorders.

14. A method of affecting a hypothalamic-related comprising:
implanting a stimulator in a target site of a hypothalamus; and
providing a stimulation signal to the stimulator to stimulate the target site to affect the hypothalamic-related condition, wherein the hypothalamic-related condition is not an eating disorder.

15. The method of claim 14, wherein the stimulator is an electrode.

16. The method of claim 14, wherein the stimulator is a catheter.

17. The method of claim 14, wherein the hypothalamic-related condition is selected from the group consisting of infertility, ovulation, irregular or painful menses, lack of lactation, hyperlactation, hypogonadism, low sperm count, impotence, baldness, and precocious puberty.

18. The method of claim 14, wherein the hypothalamic-related condition is selected from the group consisting of psychogenic polydipsia, diabetes insipidus, and SIADH.

19. The method of claim 14, wherein the hypothalamic-related condition is selected from the group consisting of heart failure, angina, hypertension, or orthostatic hypotension, pulmonary edema and asthma.

20. The method of claim 14, wherein the hypothalamic-related condition is gastroparesis or autonomic instability.

21. The method of claim 14, wherein the hypothalamic-related condition is acromegaly or dwarfism.

22. The method of claim 14, wherein the hypothalamic-related condition is selected from the group consisting of Raynaud's Syndrome, hyperhidrosis, hypothermia, hyperthermia, and facial blushing.

23. The method of claim 14, wherein the hypothalamic-related condition is sexual disorders or rage disorders.

24. The method of claim 14, wherein the hypothalamic-related condition is selected from the group consisting of insomnia, narcolepsy, lethargy, and circadian rhythms.

25. The method of claim 14, wherein the hypothalamic-related condition is selected from the group consisting of hypothyroidism, hyperthyroidism, or pro lactinoma.

26. A method of affecting a hypothalamic-related condition comprising:
implanting a stimulator in a target site of a division of a hypothalamus; and
providing a stimulation signal to the stimulator to stimulate the target site to affect the hypothalamic-related condition, wherein the hypothalamic-related condition is not an eating disorder.

27. The method of claim 26, wherein the division is a periventricular division of the hypothalamus, the periventricular division comprising the arcuate nucleus, paraventricular nucleus, periventricular nucleus, tuberoinfindibular tract, stria terminalis, dorsal longitudinal fasciculus, fomix, medial forebrain bundle, and optic chiasm.

28. The method of claim 27, wherein the hypothalamic-related condition is selected from the group consisting of chronic pain, gastroparesis, orthostatic hypotension, arrythymia, heart failure, hypertension, pulmonary edema, prolactinoma, SIADH, diabetes insipidus, lethargy, coma, acromegaly, dwarfism, infertility, ovulation, irregular or painful menses, hypogonadism, low sperm count, precocious puberty; lack of lactation, hyper-lactation, hypothyroidism, hyperthyroidism, addictions, psychogenic polydipsia, Raynaud's Syndrome, hyperthermia, fear, anxiety, sexual disorders, rage disorders, hypothermia, angina, depression, insomnia, hyperhidrosis, narcolepsy, circadian rhythms, autonomic instability, asthma, or facial blushing.

29. The method of claim 26, wherein the division is the medial division of the hypothalamus, the medial division comprising the anterior nucleus, supraoptic nucleus, preoptic nucleus, mammillary body, ventromedial nucleus, dorsomedial nucleus, posterior nucleus, substantia innominata, basal nucleus of meynert, stria terminalis, tuberoinfundibular tract, parasympathetic efferents from the hypothalamus, dorsal longitudinal fasciculus, medial forebrain bundle, fomix, mammillothalamic tract, mammillary peduncle, and pallidohypothalamic tract.

30. The method of claim 29, wherein the hypothalamic-related condition is selected from the group consisting of addiction, sexual disorders, hyperhidrosis, hyperthermia, psychogenic polydipsia, Raynaud's Syndrome, infertility, ovulation, irregular or painful menses, hypogonadism, low sperm count; precocious puberty, lack of lactation, hyper-lactation,SIADH, diabetes insipidus, gastroparesis, hypertension, angina, arrhythmia, rage disorder, depression, epilepsy, movement disorders, taste disorders, heart failure, hypothermial, insomnia, narcolepsy, hypothyroidism, hyperthyroidism, lethargy, acromegaly, dwarfism, orthostatic hypotension, chronic pain, and asthma.

31. The method of claim 26, wherein the division is the lateral division of the hypothalamus, the lateral division comprising the preoptic nucleus, supraoptic nucleus, lateral nucleus, tuberal nuclei, medial forebrain bundle, fomix, amygdalofugal fibers, parasympathetic efferents from the hypothalamus, stria terminalis, median eminence, tuberoinfundibular tract, and dorsal longitudinal fasciculus.

32. The method of claim 31, wherein the hypothalamic-related condition is selected from the group consisting of rage, depression, anxiety, epilepsy, addiction, chronic pain, infertility, ovulation, irregular or painful menses, hypogonadism, low sperm count, precocious puberty, gastroparesis, hypertension, angina, arrythymia, Raynaud's Syndrome, lack of lactation, hyper-lactation, SIADH, diabetes insipidus, hypothermia, Orthostatic hypotension, acromegaly, dwarfism, heart failure, rage disorder, depression, insomnia, lethargy, coma, hyperhidrosis, sexual disorders, psychogenic polydipsia, hypothyroidism, hyperthyroidism, pulmonary edema, asthma, chronic pain, and autonomic dysfunction.

33. A method of affecting a hypothalamic-related condition comprising:
implanting a stimulator in a nucleus of the hypothalamus; and
providing a stimulation signal to the stimulator to stimulate the nucleus of the hypothalamus to affect the hypothalamic-related condition, wherein the hypothalamic-related condition is not an eating disorder.

34. The method of claim 33, wherein the nucleus of the hypothalamus is selected from the group consisting of the paraventricular nucleus, preoptic nucleus, supraoptic nucleus, anterior nucleus, suprachiasmatic nucleus, ventromedial nucleus, dorsomedial nucleus, arcuate nucleus, lateral nucleus, posterior nucleus, substantia innominata, basal nucleus of meynert, and mammillary body.

35. The method of claim 33, wherein the nucleus is an anterior nucleus and the hypothalamic-related condition is selected from the group consisting of addiction, fear, anxiety, sexual disorders, hyperhidrosis, hyperthermia, psychogenic polydipsia, Raynaud's Syndrome, infertility, ovulation, irregular menses or painful menses, hypogonadism, low sperm count, impotence, baldness and precocious puberty.

36. The method of claim 33, wherein the nucleus is an arcuate nucleus and the hypothalamic-related condition is selected from the group consisting of lack of lactation, hyper-lactation, chronic pain, infertility, ovulation, irregular menses, painful menses, hypogonadism, low sperm count, impotence, baldness and precocious puberty.

37. The method of claim 33, wherein the nucleus is a parventricular nucleus and the hypothalamic-related condition is selected from the group consisting of chronic pain, gastroparesis, Orthostatic hypertension, arrythymia, heart failure; hypothyroidism, hyperthyroidism, hypertension, pulmonary edema; no lactation, hyper-lactation, prolactinoma, SIADH, diabetes insipidus, lethargy, coma, acromegaly, and dwarfism.

38. The method of claim 33, wherein the nucleus in a supraoptic nucleus and the hypothalamic-related condition is selected from the group consisting of addictions, no lactation, hyper-lactation, SLADH, diabetes insipidus, gastroparesis, hypertension, angina, arrhythmia, impotence, and Raynaud's Syndrome.

39. The method of claim 33, wherein the nucleus is a suprachiasmatic nucleus and the hypothalamic-related condition is selected from the group consisting of circadian rhythms, insomnia, and narcolepsy.

40. The method of claim 33, wherein the nucleus is a preoptic nucleus and the hypothalamic-related condition is selected from the group consisting of rage, depression, anxiety, epilepsy, addiction, chronic pain, infertility, ovulation, irregular or painful menses, hypogonadism, low sperm count, precocious puberty; gastroparesis, hypertension, angina, arrythymia, impotence, baldness, and Raynaud's Syndrome

41. The method of claim 33, wherein the nucleus is a lateral nucleus and the hypothalamic-related condition is selected from the group consisting of rage disorder; Epilepsy, anxiety, addictions, obsessive-compulsive disorder, heart failure, hypothermia, insomnia, narcolepsy, gastroparesis, hypertension, angina, arrhythmia, Raynaud's Syndrome, hyperhidrosis, hyperthermia, psychogenic polydipsia.

42. The method of claim 33, wherein the nucleus is a mammillary body and the hypothalamic-related condition is selected from the group consisting of epilepsy, chronic pain, gastroparesis, hypertension, and angina.

43. The method of claim 33, wherein the nucleus is a ventromedial nucleus and the hypothalamic-related condition is selected from the group consisting of chronic pain, addictions, rage disorder, movement disorders, psychiatric disorders, infertility, ovulation, irregular or painful menses, hypogonadism, low sperm count, impotence, baldness and precocious puberty.

44. The method of claim 33, wherein the nucleus is a dorsomedial nucleus and the hypothalamic-related condition is selected from the group consisting of epilepsy, chronic pain, depression, rage, hypothyroidism, and hyperthyroidism.

45. The method of claim 33, wherein the nucleus is a posterior nucleus and the hypothalamic-related condition is selected from the group consisting of chronic pain, taste disorder, rage disorder, hypertension, anxiety, heart failure, hypothermia, insomia, narcolepsy, precocious puberty, and hypogonadism.

46. The method of claim 33, wherein the nucleus is a substantia innominata and the hypothalamic-related condition is taste disorder.

47. The method of claim 33, wherein the nucleus is a basal nucleus of meynert and the hypothalamic-related condition is Alzheimer's disease or dementia.

48. A method of affecting a hypothalamic-related condition comprising:
implanting a stimulator in a target site of a hypothalamus; and
providing a stimulation signal to the stimulator to stimulate the target site to modulate the synthesis or release of a hypothalamic or pituitary product.

49. The method of claim 48, wherein the hypothalamic or pituitary product is a hormone.

50. A method of affecting a hypothalamic-related condition comprising:
implanting a stimulator in a target site of a pituitary gland; and
providing a stimulation signal to the stimulator to stimulate the target site to modulate the synthesis or release of a hypothalamic or pituitary product.

51. The method of claim 50, wherein the hypothalamic or pituitary product is a hormone.

52. A method of affecting a hypothalamic-related condition comprising:
implanting a stimulator in communication with a hypothalamic-related target site;
detecting a physiological activity of the body associated with the hypothalamic-related condition to generate a sensor signal;
providing a stimulation signal to the stimulator in response to the sensor signal; and
stimulating the target site to affect the hypothalamic-related condition.
